(19) United States
(12) Patent Application Publication
(10) Pub. No.: US 2020/0191759 A1
(43) Pub. Date: Jun. 18, 2020

(54) CALIBRATING DEVICE FOR AUTOMATICALLY CALIBRATING DATA OF MEASURING INSTRUMENT AND METHOD THEREOF

(71) Applicant: CAMEO INFOTECH INC., Taipei City (TW)

(72) Inventors: CHENG HAN WU, TAIPEI CITY (TW); PO HUAN CHIU, TAIPEI CITY (TW); PEI YUN TSAI, NANTOU CITY (TW)

(21) Appl. No.: 16/695,792

(22) Filed: Nov. 26, 2019

(30) Foreign Application Priority Data
Nov. 28, 2018 (CN) 201811439342.8

(51) Int. Cl. G01N 33/00; G01N 30/72; G01N 30/86

(52) U.S. Cl. CPC G01N 33/0008; G01N 2030/025; G01N 30/8668; G01N 30/7206

(57) ABSTRACT

An automatic measuring instrument data correction device and method related to the technical field of environmental measuring instrument are disclosed. The device includes a data reading and converting module obtaining readable data having time information and generated by the measuring instrument; an automatic data correction module comparing the preset standard spectrum with the readable data having time information, executing data correction according to a comparison result. Therefore, the measurement result, which is generated by the measuring instrument, can be corrected to greatly reduce labor cost and time cost, and further improve production efficiency.

### BACKGROUND OF THE INVENTION

### 1. Field of the Invention

The present invention relates to a technical field of environmental measuring instrument, and more particularly to a calibrating device for automatically calibrating data of measuring instrument and a method thereof

### 2. Description of the Related Art

Existing measuring instruments need to be corrected and calibrated, mainly because there are many uncertain factors affecting measurement results of measuring instruments in use, for example, the factors include wear of measuring instruments in use, ambient temperature, pressure environment, human factors, and so on. These uncertain factors may cause variations and errors in the measurement results obtained by the measuring instrument.

Data drift is a data error problem often occurring in a measuring instrument. The data drift occurs because of environmental factors during the entire operation period of the measuring instrument and will permanently change the original design, structure or performance of the measuring instrument.

In the environmental industry, online monitors that detect volatile organic compounds (VOCs) often have data drift problems. In order to solve this problem, an existing solution is that a user operates the online monitor to generate measurement values first and compare the measurement values with the standard spectrum one by one, and the user then corrects the measurement values manually if finding offsets or drifts existing in the measurement values. Obviously, it consumes a lot of manpower.

### SUMMARY OF THE INVENTION

An objective of the present invention is to provide a calibrating device for automatically calibrating data of measuring instrument and a method thereof, in order to automatically perform data calibration, decrease the labor cost, and increase the calibration efficiency.

The present invention provides a technical solution below.

A calibrating device for automatically calibrating data of measuring instrument comprises a data reading and converting module configured to obtain readable data having time information and generated by a measuring instrument; an automatic data correction module configured to compare the preset standard spectrum with the obtained readable data having time information, and execute corresponding data correction according to a comparison result.

In the technical solution, the measurement result, which is generated by the measuring instrument, is corrected in order to greatly reduce labor cost and time cost, thereby increasing production efficiency.

The technical solution further comprises a data report module configured to generate an instrument data report.

The technical solution further comprises an automatic operation module configured to control the measuring instrument to generate the instrument data report.

In the technical solution, the instrument data report can be generated by the measuring instrument or the data report module upon a practical demand.

In the technical solution, the data reading and converting module, which obtains the readable data having time information and generated by the measuring instrument, further comprises a data reading and converting module configured to obtain raw data generated by the measuring instrument, and when the raw data is a comma-separated value (CSV) file, the CSV file can be used as the readable data having time information.

In the technical solution, the raw data is converted into the CSV file readable for the automatic data correction module, so as to lay the foundation for subsequent data correction.

In the technical solution, the data reading and converting module, which obtains the readable data having time information and generated by the measuring instrument, performs following operations: the data reading and converting module obtains the raw data generated by the measuring instrument, and when the raw data is a CSV file but not directly readable, the data reading and converting module removes abnormal values in the CSV file, and uses the CSV file with abnormal value removed, as the readable data having time information.

In the technical solution, removing the abnormal values ensures the CSV file with the abnormal value removed to be a readable file for the automatic data correction module, so as to lay the foundation for data correction.

Further, the data reading and converting module, which obtains the readable data having time information and generated by the measuring instrument, performs following operations: the data reading and converting module obtains the raw data generated by the measuring instrument, and when the raw data is a screenshot of spectrum data generated by the measuring instrument, the data reading and converting module converts the screenshot into the readable data having time information.

In the technical solution, the readable data can be obtained from conversion of the screenshot, so that the automatic data correction module can smoothly read data.

Further, the manner of using the data reading and converting module to obtain the raw data generated by the measuring instrument can be one of following manners: the data reading and converting module receives the raw data transmitted by the measuring instrument; the data reading and converting module downloads the raw data, which is uploaded by the measuring instrument, from the industrial personal computer; the data reading and converting module downloads the raw data, which is uploaded by the measuring instrument, from the platform server.

In the technical solution, there are various manners of obtaining the raw data and at least one of the manners can be selected upon actual requirement; no matter which manner is selected, the raw data can be automatically obtained by the data reading and converting module without manual intervention.

The technical solution further comprises an automatic operation module is controlled by automatic data correction module to automatically activate the measuring instrument to generate the raw data.

In the technical solution, the measuring instrument can be controlled to turn on/off, so as to reduce manual intervention and improve automation.

The present invention further provides an automatic measuring instrument data correction method comprising steps of: obtaining readable data having time information and generated by a measuring instrument; comparing a preset standard spectrum with the readable data having time information, and executing corresponding data correction according to a comparison result.

In the technical solution, correcting the measurement result generated by the measuring instrument can greatly reduce labor cost and time cost, thereby increasing production efficiency.

The method further comprises a step of generating an instrument data report.

The method further comprising a step of controlling the measuring instrument to generate the instrument data report.

In the method, the step of obtaining the readable data having time information and generated by the measuring instrument further comprises: obtaining raw data generated by the measuring instrument; when the raw data is a CSV file, using the CSV file as the readable data having time information.

In the method, the step of obtaining the readable data having time information and generated by the measuring instrument further comprises: obtaining raw data generated by the measuring instrument; when the raw data is a CSV file but not directly readable, removing abnormal values in the CSV file, and using the CSV file with the abnormal value removed as the readable data having time information.

In the method, the step of obtaining the readable data having time information and generated by the measuring instrument further comprises: obtaining raw data generated by the measuring instrument; when the raw data is a screenshot of spectrum data generated by the measuring instrument, converting the screenshot into the readable data having time information.

In the method, the manners of obtaining the raw data generated by the measuring instrument can be one of manners below: receiving the raw data transmitted by the measuring instrument; downloading the raw data, which is uploaded by the measuring instrument, from the industrial personal computer; downloading the raw data, which is uploaded by the measuring instrument, from the platform server.

In the technical solution, the method further comprises a step of controlling the measuring instrument to automatically start generating the raw data.

Compared with conventional technology, the calibrating device for automatically calibrating data of measuring instrument and method of the present invention have the beneficial effects below.

The calibrating device for automatically calibrating data of measuring instrument and method of the present invention can correct the measurement result generated by the measuring instrument, so as to greatly reduce labor cost and time cost, and further improve production efficiency.

### BRIEF DESCRIPTION OF THE DRAWINGS

The structure, operating principle and effects of the calibrating device for automatically calibrating data of measuring instrument and method of the present invention will be described in detail by way of various embodiments which are illustrated in the accompanying drawings.

FIG. 1 is a schematic structural view of an embodiment of a calibrating device for automatically calibrating data of measuring instrument, according to the present invention.

FIG. 2 is a schematic structural view of an embodiment of a preset standard spectrum of the present invention.

FIG. 3 is a schematic structural view of another embodiment of a calibrating device for automatically calibrating data of measuring instrument, according to the present invention.

FIG. 4 is a flowchart of an embodiment of an unmanned automatic data correction operation of a gas chromatography online VOC monitor of the present invention.

FIG. 5 is a flowchart of an embodiment of an unmanned automatic data correction operation of a gas mass spectrometry online VOC monitor of the present invention.

FIG. 6 is a flowchart of an embodiment of a data drift determination operation of the present invention.

FIG. 7 is a structural view of an embodiment of an unmanned automatic data correction system of the gas chromatography online VOC monitor of the present invention.

FIG. 8 is a structural view of an embodiment of an unmanned automatic data correction system of a gas mass spectrometry online VOC monitor of the present invention.

FIG. 9 is a flowchart of an embodiment of a calibrating method for automatically calibrating data of measuring instrument , according to the present invention.

FIG. 10 is a flowchart of another embodiment of a calibrating method for automatically calibrating data of measuring instrument, according to the present invention.

### DETAILED DESCRIPTION OF THE PREFERRED EMBODIMENTS

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims. These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts.

It is to be acknowledged that, although the terms ‘first’, ‘second’, ‘third’, and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the word “comprise”, “include” and “have”, and variations such as “comprises”, “comprising”, “includes”, “including”, “has” and “having” will be acknowledged to imply the inclusion of stated elements but not the exclusion of any other elements. As used herein, the singular forms “a”, “an” and “the” are intended to include the plural forms as well, unless the context clearly indicates otherwise.

In environmental industry, a main process cycle of a sampling analysis operation of a VOC detecting measuring instrument can be separated into five steps: a sampling step, a compound concentration step, an injection step, a spectrum analysis step, and a checking step. In the spectrum analysis step, a flame ionization detector generates an electric signal, and signal strength of the electric signal is positively proportional to flow of the sample component from an analytical column. Next, the electric signal is digitized and then transmitted to a CPU card, and a microprocessor is used to perform data aggregation, calculation of compound mass or concentration, and peak identification. Eventually, the compound is identified according to a retention time of compound, and the concentration is calculated with reference to standard compound analysis.

For example, the measuring instrument can detect ethane and ethylene, and generally, the retention time of ethylene is in range of 20 seconds to 30 seconds, and the retention time of ethane is in a range of 0 second to 15 seconds. The measuring instrument is determined to occur data drift phenomenon in a condition that no gas concentration is detected from 0second to 15 seconds, and the gas (ethane) concentration is just detected between 20 seconds and 30 seconds, and another gas concentration is detected between 40 seconds and 50 seconds, and at this time, the measuring instrument determines that absence of ethane is detected, and incorrectly defines the detected gas as other species, and it causes wrong detection result. Therefore, the data generated by the measuring instrument needs to be corrected. It should be noted that the aforementioned description just interprets a basic principle, and other actual condition is not described in detail.

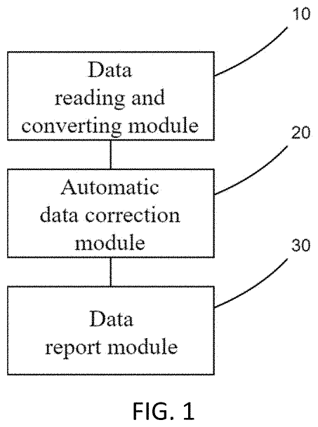

The present invention provides an embodiment of a calibrating device for automatically calibrating data of measuring instrument. The calibrating device for automatically calibrating data of measuring instrument, which is abbreviated as a data calibration host hereafter for convenience, can be implemented by a computer such as an Apple PC, a Windows PC, and so on. As shown in FIG. 1, the data calibration host comprises a data reading and converting module 10 configured to obtain readable data having time information and generated by the measuring instrument; an automatic data correction module 20 configured to compare the preset standard spectrum with the obtained readable data having time information, and then execute corresponding data correction according to a comparison result.

Particularly, an objective of this embodiment is to replace the conventional manual correction process by an automatic process executed by a computer, so as to decrease labor cost. Related program can be installed in the computer to implement the automatic data calibrating function.

The comparison and the correction processes are fully completed by the computer, and when the raw data generated by the measuring instrument is the readable data having time information readable for the computer, it is not necessary to convert the raw data, so the data reading and converting module 10 can directly obtain the raw data.

For example, in regard to a gas chromatography measuring instrument, the raw data generated by the gas chromatography measuring instrument is a comma-separated values (CSV) file; sometimes, the CSV file is directly readable for the automatic data correction module 20, for example, when the CSV does not have any abnormal value. Particularly, during the process in which the data reading and converting module 10 obtains the readable data having time information and generated by the measuring instrument, the data reading and converting module 10 obtains the raw data generated by the measuring instrument, and when the raw data is the CSV file and is directly readable for the automatic data correction module, the CSV file is used as the readable data having time information.

In most cases, the raw data generated by the measuring instrument is not the data identifiable for the computer, and the data must be converted. There are two manners to obtain the readable data having time information and generated by the measuring instrument.

In the first manner, the raw data generated by the gas chromatography measuring instrument may have abnormal values, that is, the CSV file may have abnormal values such as 0 value, empty value, and so on, and the abnormal values cause the CSV file to be unidentifiable for the automatic data correction module 20.

Therefore, during a particular process in which the data reading and converting module 10 obtains the readable data having time information and generated by the measuring instrument, the data reading and converting module 10 obtains the raw data generated by the measuring instrument, and when the raw data is a CSV file but not directly-readable for the automatic data correction module 20, the data reading and converting module 10 removes abnormal values in the CSV file, and the CSV file with abnormal value removed is used as the readable data having time information.

In the second manner, for the raw data generated by a gas mass spectrometry measuring instrument, a screen capture program is used to capture a screenshot of spectrum data generated by data reading software of the gas mass spectrometry measuring instrument, and image to text software is required to convert the screenshot of the spectrum data into the data readable for the automatic data correction module 20.

Therefore, during a particular process in which the data reading and converting module 10 obtains the readable data having time information and generated by the measuring instrument, the data reading and converting module 10 obtains the raw data generated by the measuring instrument, and when the raw data is the screenshot of the spectrum data generated by the measuring instrument, the data reading and converting module 10 converts the screenshot into the readable data having time information.

In practical use, one of the above-mentioned manners can be selected according to an actual condition, to obtain the readable data having time information, for sequential automatic correction.

The manner of enabling the data reading and converting module 10 to obtain the raw data generated by the measuring instrument can be one of following manners.

In the first manner, the data reading and converting module 10 receives the raw data transmitted by the measuring instrument.

Particularly, when the measuring instrument is in direct communication and connection with the data calibration host, the measuring instrument can directly transmit the raw data to the data calibration host.

In the second manner, the data reading and converting module 10 downloads the raw data, which is uploaded by the measuring instrument, from the industrial personal computer.

Particularly, a measuring instrument is in connection and communication with an industrial personal computer (IPC), such as through RS232 communicative connection, the industrial personal computer can obtain data through RS232 and store the data in the memory thereof, so that the data calibration host can download, through a FTP manner, the raw data of the measuring instrument from the industrial personal computer.

In the third manner, the data reading and converting module 10 downloads the raw data, which is uploaded by the measuring instrument, from the platform server.

Particularly, a measuring instrument is in direct communication and connection with a platform server, the measuring instrument transmits the raw data to the platform server for storage, so that the data calibration host can directly download the raw data, from the platform server, to the memory thereof

The automatic data correction module 20 compares a preset standard spectrum with the obtained readable data having time information, and when the automatic data correction module 20 finds out data drift, the automatic data correction module 20 performs data correction; when no data drift is found, the automatic data correction module 20 does not perform data correction. Generally, the data drift of the readable data having time information can be determined according to volatility time of the species starting to volatile earliest.

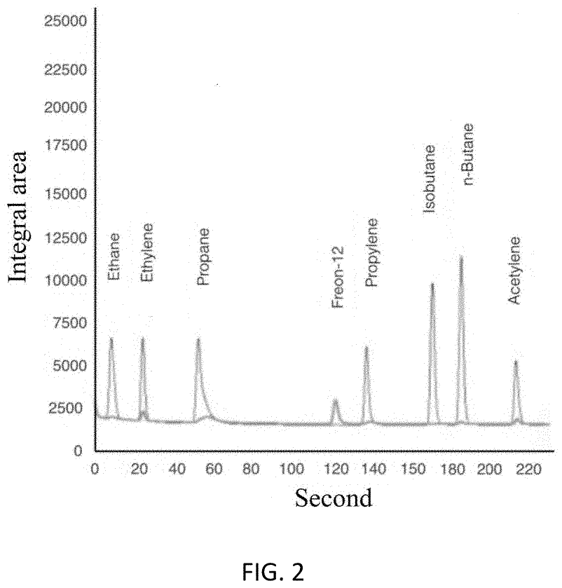

The preset standard spectrum can be set according to the species to be actually detected by the measuring instrument. For example, FIG. 2 shows a preset standard spectrum.

Optionally, the calibrating device for automatically calibrating data of measuring instrument further comprises a data report module 30 configured to generate the instrument data report.

Particularly, after the readable data having time information is corrected completely, the data calibration host outputs the instrument data report for a user to review. The instrument data report can be the corrected data report, or a data drift determination report generated according to the correction process.

In this embodiment, the calibrating device for automatically calibrating data of measuring instrument automatically corrects the measurement result generated by the measuring instrument, so as to greatly reduce labor cost and time cost, and further improve production efficiency.

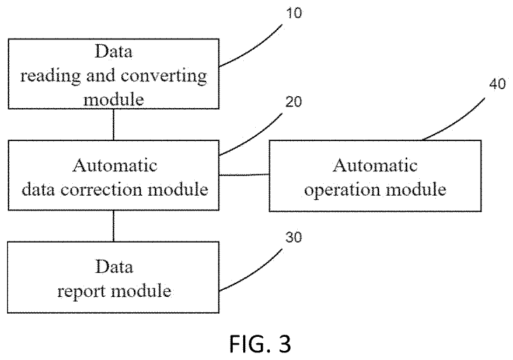

In another embodiment of the present invention, as shown in FIG. 3, an calibrating device for automatically calibrating data of measuring instrument comprises a data reading and converting module 10 configured to obtain the readable data having time information and generated by the measuring instrument; an automatic data correction module 20 configured to compare the preset standard spectrum with the obtained readable data having time information, and execute corresponding data correction according to a comparison result; an automatic operation module 40 configured to control the measuring instrument to generate the instrument data report.

Particularly, after the data correction is performed completely, the automatic operation module can control the measuring instrument to generate the instrument data report. For example, the data calibration host can simulate a cursor simulator to operate software of controlling the measuring instrument, so as to control the measuring instrument to generate the instrument data report.

Optionally, the automatic operation module 40 is further controlled by the automatic data correction module 20, to automatically activate the measuring instrument to generate the raw data.

Particularly, the data calibration host can execute a system call to simulate a cursor simulator 401 of a computer, to automatically activate the software corresponding to the measuring instrument to start operating without manual intervention, thereby generating the raw data.

Optionally, there mainly are three manners of obtaining the readable data having time information and generated by the readable data having time information.

The first manner is for the gas chromatography measuring instrument; during the particular process in which the data reading and converting module 10 obtains the readable data having time information and generated by the measuring instrument, the data reading and converting module 10 obtains the raw data generated by the measuring instrument, and when the raw data is a CSV file and directly readable, the CSV file is used as the readable data having time information.

The second manner is for the gas chromatography measuring instrument; during the particular process in which the data reading and converting module 10 obtains the readable data having time information and generated by the measuring instrument, the data reading and converting module 10 obtains the raw data generated by the measuring instrument, and when the raw data is a CSV file but not directly readable, the data reading and converting module 10 removes abnormal values in the CSV file, and the CSV file with abnormal value removed is used as the readable data having time information.

The third manner is for the gas mass spectrometry measuring instrument; during the particular process in which the data reading and converting module 10 obtains the readable data having time information and generated by the measuring instrument, the data reading and converting module 10 can obtain the raw data generated by the measuring instrument, and when the raw data is a screenshot of the spectrum data generated by the measuring instrument, the data reading and converting module 10 converts the screenshot into the readable data having time information.

In practical use, at least one of the above-mentioned manners can be used according to the actual condition, to obtain the readable data having time information for sequential automatic correction.

The manner of enabling the data reading and converting module to obtain the raw data generated by the measuring instrument can be one of following manners.

In the first manner, the data reading and converting module 10 receives the raw data transmitted by the measuring instrument.

In the second manner, the data reading and converting module 10 downloads the raw data, which is uploaded by the measuring instrument, from the industrial personal computer.

In the third manner, the data reading and converting module 10 downloads the raw data, which is uploaded by the measuring instrument, from the platform server.

Optionally, the calibrating device for automatically calibrating data of measuring instrument can comprise the data report module 30 configured to generate the instrument data report.

Upon practical demand, at least one of the measuring instrument and the data calibration host can be selected to generate the instrument data report.

The parts of this embodiment the same as that of previous embodiment can be referred to the previous embodiment, so detailed descriptions are not repeated herein.

In this embodiment, the calibrating device for automatically calibrating data of measuring instrument can automatically control the measuring instrument to work without manual intervention, so as to improve efficiency of the measurement result correction, and decrease labor cost and time cost.

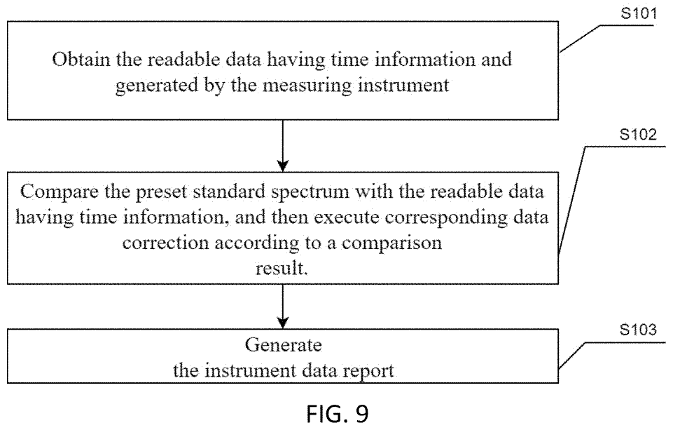

FIG. 9 shows an embodiment of an automatic measuring instrument data correction method of the present invention. The automatic measuring instrument data correction method comprises following steps.

In a S101, a data calibration host obtains the readable data having time information and generated by the measuring instrument.

In a S102, the data calibration host compares the preset standard spectrum with the readable data having time information, and then executes corresponding data correction according to a comparison result.

Particularly, the objective of this embodiment is to replace the conventional manual correction process by an automatic process executed by a computer, so as to decrease labor cost. The related program can be installed in the computer to implement the automatic data calibrating function.

The comparison and correction processes are fully completed by the computer, and when the raw data generated by the measuring instrument is the readable data having time information for the computer, it does not need to convert the raw data, and the computer can directly obtain and use the raw data.

For example, the raw data generated by a gas chromatography measuring instrument is a CSV file which is sometimes directly readable for computer, for example, in a condition that there is no abnormal value in the CSV file. Therefore, during the particular process in which the data reading and converting module 10 obtains the readable data having time information and generated by the measuring instrument, the data reading and converting module 10 can obtain the raw data generated by the measuring instrument, and when the raw data is a CSV file and is directly readable, the CSV file is used as the readable data having time information.

In most cases, the raw data generated by the measuring instrument is not identifiable for the computer, so the raw data must be converted. There are two manners for the data calibration host to obtain the readable data having time information and generated by the measuring instrument.

In the first manner, the raw data generated by a gas chromatography measuring instrument may have abnormal values, that is, the CSV file may have abnormal values, such as 0 value, empty value, and so on, and the abnormal values cause the CSV file to be unidentifiable for the automatic data correction module 20.

Particularly, the method for the data calibration host to obtain the readable data having time information and generated by the measuring instrument, includes steps of: obtaining the raw data generated by the measuring instrument; when the raw data is a CSV file but not directly readable, removing abnormal values in the CSV file, and using the CSV file with the abnormal value removed as the readable data having time information.

In the second manner, for the raw data generated by a gas mass spectrometry measuring instrument, a screen capture program is used to capture a screenshot of the spectrum data generated by data reading software of the gas mass spectrometry measuring instrument, and image to text software is required to convert the spectrum data into the data readable for the automatic data correction module 20.

Therefore, the manner of obtaining the readable data having time information and generated by the measuring instrument can comprise steps of obtaining, by the data calibration host, the raw data generated by the measuring instrument; when the raw data is the screenshot of spectrum data generated by the measuring instrument, converting the screenshot into the readable data having time information.

In practical use, at least one of the above-mentioned manners can be used according to the actual condition, to obtain the readable data having time information for sequential automatic correction.

The manner of obtaining the raw data generating the measuring instrument can be one of following manners.

In the first manner, the data calibration host receives the raw data transmitted from the measuring instrument.

Particularly, when the measuring instrument is in direct communication and connection with the data calibration host, the measuring instrument can directly transmit the raw data to the data calibration host.

In the second manner, the data calibration host downloads the raw data, which is uploaded by the measuring instrument, from the industrial personal computer.

Particularly, the measuring instrument is in communication and connection with an industrial personal computer, such as through RS232 communicative connection, the industrial personal computer can capture, through RS232, data and store the data in the memory thereof, and the data calibration host downloads the raw data of the measuring instrument from the industrial personal computer, through a FTP manner.

In the third manner, the data reading and converting module downloads the raw data, which is uploaded by the measuring instrument, from the platform server.

Particularly, a measuring instrument is in direct communication and connection with a platform server, the measuring instrument transmits the raw data to the platform server for storage, and the data calibration host can directly download the raw data to the memory thereof, from the platform server.

The data calibration host can compare the preset standard spectrum with the obtained readable data having time information, and when the data calibration host finds data drift in the data, the data calibration host performs data correction; when there is no data drift, the data calibration host does not execute data correction. The data drift of the readable data having time information can be determined according to the volatility time of the species starting to volatile earliest.

The preset standard spectrum can be set according to the species detected by the measuring instrument actually; for example, FIG. 2 shows a preset standard spectrum.

Optionally, the automatic measuring instrument data correction method can comprise a step S103 of generating the instrument data report.

Particularly, after the readable data having time information is corrected completely, the data calibration host outputs the instrument data report for a user to review.

The instrument data report can be the corrected data report, or the data drift determination report generated according to the correction process.

In this embodiment, the calibrating device for automatically calibrating data of measuring instrument can automatically correct the measurement result generated by the measuring instrument, so as to greatly reduce labor cost and time cost, and further improve production efficiency.

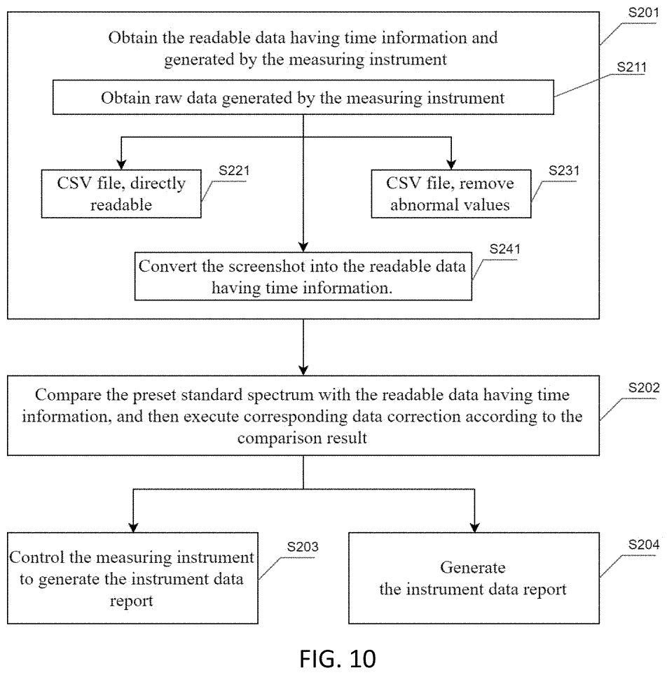

FIG. 10 shows an embodiment of an automatic measuring instrument data correction method of the present invention. The embodiment of the method comprises following steps.

In a step S201, the data calibration host obtains the readable data having time information and generated by the measuring instrument.

In a step S202, the data calibration host compares the preset standard spectrum with the readable data having time information, and then executes corresponding data correction according to the comparison result.

In a step S203, the data calibration host controls, by using the cursor simulator, the measuring instrument to generate the instrument data report.

Particularly, after the data correction is performed completely, the measuring instrument is controlled to generate the instrument data report. Particularly, the data calibration host can simulate the cursor simulator 401 to operate the software of controlling the measuring instrument to generate the instrument data report.

Optionally, the automatic measuring instrument data correction method can comprise a step of controlling, by the data calibration host, the measuring instrument to automatically start to generate the raw data.

Particularly, the data calibration host can execute a system call to simulate a cursor simulator of a computer to automatically activate the software corresponding to the measuring instrument to start operation, so as to generate the raw data without manual intervention.

Optionally, there mainly are three manners of obtaining the readable data having time information and generated by the readable data.

The first manner is for a gas chromatography measuring instrument, and the step S201 of obtaining the readable data having time information and generated by the measuring instrument can include steps S211 and S221.

in a step S211, the raw data generated by the measuring instrument is obtained;

in a step S221, when the raw data is a CSV file and directly-readable, the CSV file can be used as the readable data having time information.

The second manner is for a gas chromatography measuring instrument, and the step S201 of obtaining the readable data having time information and generated by the measuring instrument can comprise steps S211 and S231.

in the S211, the raw data generated by the measuring instrument is obtained;

in a S231, when the raw data is a CSV file but not directly-readable, abnormal values in the CSV file are removed, the CSV file with abnormal value removed is used as the readable data having time information.

The third manner is for the gas mass spectrometry measuring instrument, and the step S201 of obtaining the readable data having time information and generated by the measuring instrument can comprise steps S211 and S241.

In the S211, the raw data generated by the measuring instrument is obtained;

In a S241, when the raw data is the screenshot of spectrum data generated by the measuring instrument, the screenshot is converted into the readable data having time information.

In practical use, at least one of the above-mentioned manners can be used according to the actual condition, to obtain the readable data having time information for sequential automatic correction.

The manner of obtaining the raw data generated by the measuring instrument, can be one of following manners.

In the first manner, the data calibration host can receive the raw data transmitted by the measuring instrument.

In the second manner, the data calibration host can download the raw data, which is uploaded by the measuring instrument, from the industrial personal computer.

In the third manner, the data calibration host can download the raw data, which is uploaded by the measuring instrument, from the platform server.

Optionally, the automatic measuring instrument data correction method can further comprise a step S204 of generating the instrument data report by data calibration host. Upon practical demand, the user can select at least one of the measuring instrument or the data calibration host to generate the instrument data report.

The parts of this embodiment the same as that of previous embodiment can be referred to the previous embodiment, so detailed descriptions are not repeated herein.

In this embodiment, the calibrating device for automatically calibrating data of measuring instrument can automatically control the measuring instrument to work without manual intervention, so as to improve the efficiency of the measurement result correction, and decrease labor cost and time cost.

Some practical use examples are illustrated in following paragraphs.

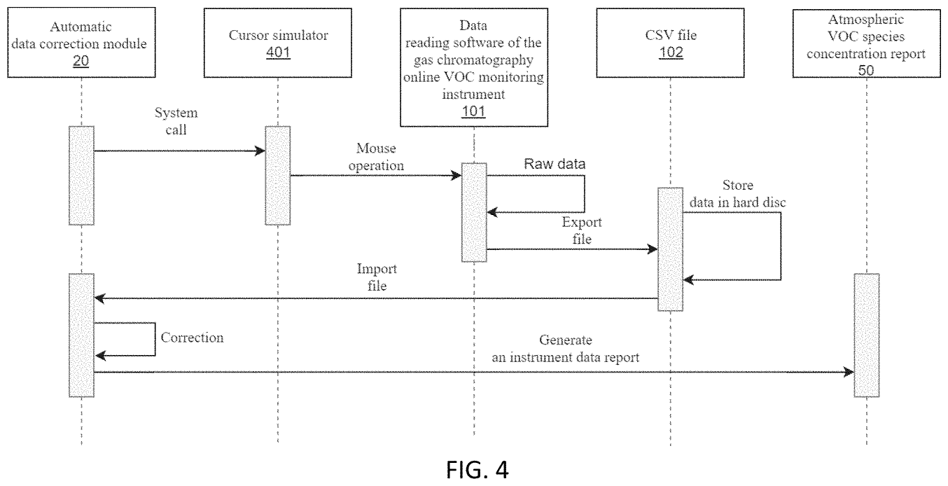

FIG. 4 shows a first practical use example in which a gas chromatography online VOC monitor serves as a measuring instrument. The process in which the data calibration host corrects the generated measurement result and generates report is described below. The automatic data correction module 20 of the data calibration host executes a system call to simulate a cursor simulator 401, which is the automatic operation module, in a computer, and the cursor simulator 401 executes the control software to control the gas chromatography online VOC monitor to start operation, and the cursor simulator 401 activates the data reading software of the gas chromatography online VOC monitor 101 without manual intervention, that is, the data reading and converting module is automatically activated. After the data reading software is activated, a file export process recorded in advance is performed to operate the data reading software of the online VOC monitor 101, to export the CSV file 102 with the gas chromatography VOC values, and store the CSV file 102 in a hard disc of the data calibration host. Next, through the process, the automatic data correction module 20 reads the CSV file, and after the automatic data correction module 20 recalculates and corrects the input data according to the preset standard spectrum or a preset algorithm, the instrument data report can be generated, that is, it is equivalent that the data report module generates the instrument data report; for example, the instrument data report can be an atmospheric VOC species concentration report 50.

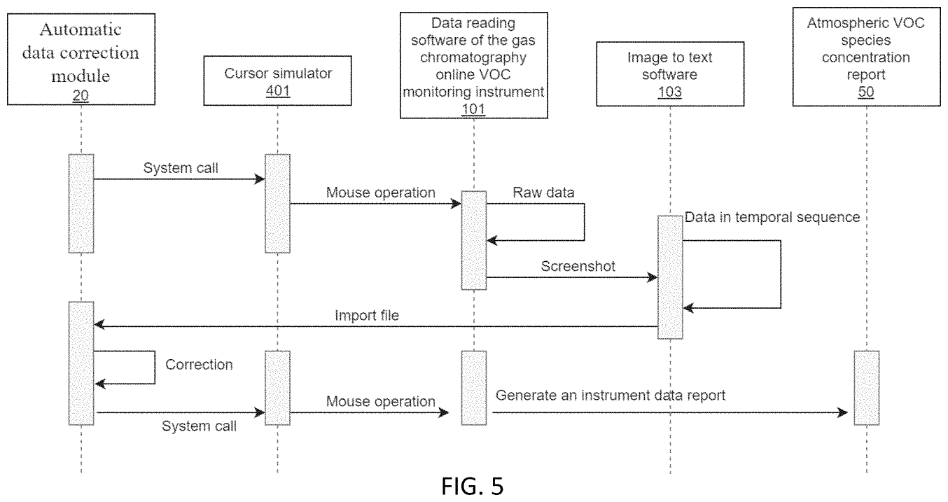

FIG. 5 shows a second practical use example in which a gas mass spectrometry online VOC monitor serves as a measuring instrument. The process in which the data calibration host corrects the generated measurement result and generate report is described below. The automatic data correction module 20 of the data calibration host executes a system call to simulate a cursor simulator 401, which is the automatic operation module, in a computer, in order to activate the data reading software 101 of the gas mass spectrometry online VOC monitor without manual intervention. After the data reading software 101 is activated, the image to text software 103 uses the screenshot software to capture screenshots of the instrument screen of the gas mass spectrometry online VOC monitor and the numerical spectrum of the software, and then convert the screenshot into data in temporal sequence. The data is transmitted to the automatic data correction module 20, and after the automatic data correction module 20 recalculates and corrects the input value according to the preset standard spectrum or preset algorithm, the automatic data correction module 20 uses the cursor simulator 401 to operate the gas mass spectrometry online VOC monitor, to generate the instrument data report compliant with the self-defined data, and the instrument data report is the corrected instrument data report.

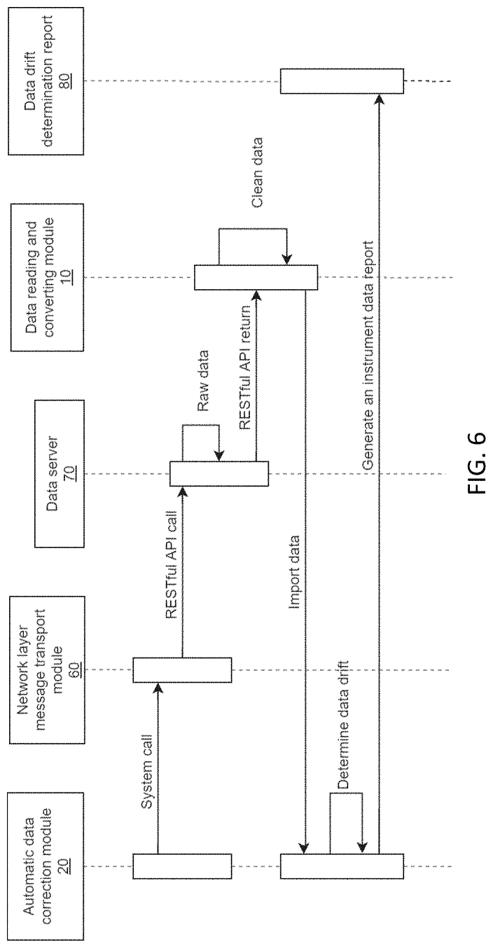

FIG. 6 shows a third practical use example in which the automatic data correction module 20 operates a data server 70 through a network layer message transport module 60. The network layer message transport module 60 can be a RESTful API call. The data server 70 transmits data to the data reading and converting module through network, and the data reading and converting module converts and cleans the data, and then transmits the cleaned data to the automatic data correction module 20. The automatic data correction module 20 performs data drift determination on the obtained data according to the preset standard spectrum or preset algorithm, and the data report module then generates the instrument data report, which is a data drift determination report. In this example, the data calibration host obtains the raw data from the data server 70, and converts, cleans and corrects the raw data, and issues the data drift determination report 80 eventually.

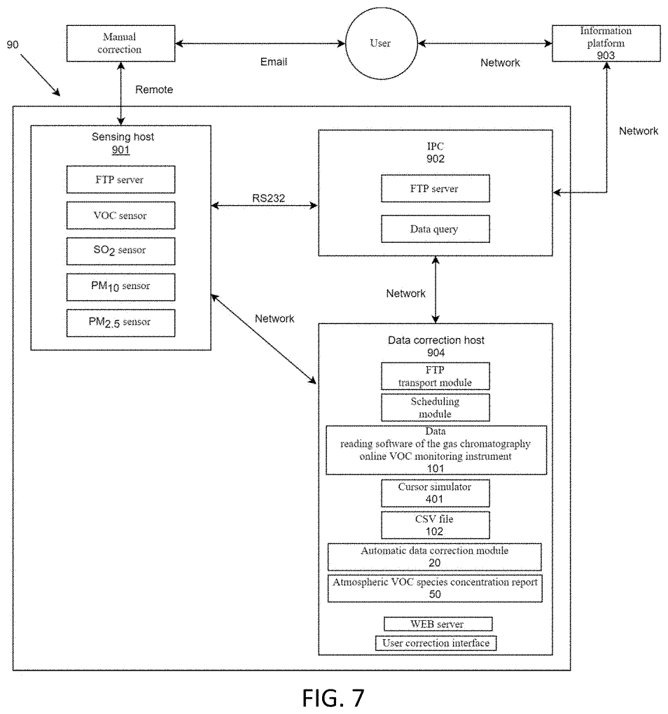

FIG. 7 shows a structural view of an unmanned automatic data correction system of the gas chromatography online VOC monitor 90 of a fourth practical use example. A sensing host 901 is mainly used to sense various species concentrations in atmosphere, and generate various species concentration reports. In conventional manner, the industrial personal computer 902 reads data through RS232 and stores the data, and the industrial personal computer 902 then uploads the data to an information platform 903, the client can query the real-time generated data through the information platform 903. However, the data generated by the sensing host 901 may have data drift, and must be corrected manually to ensure correction of the values thereof. Conventionally, the manual correction is performed by remotely logging into the sensing host 901 to read file, and obtaining and correcting the data manually, and then mailing the corrected data to the client. The unmanned automatic data correction system of the gas chromatography online VOC monitor 90 includes a data calibration host 904 additionally. The data calibration host 904 is used to replace the manual correction. The data calibration host 904 uses an FTP transport module to download raw data, which is generated by the sensing host 901, through network. The cursor simulator 401 of the unmanned automatic data correction system can simulate a person's operating behavior to use the data reading software 101 of the gas chromatography online VOC monitor to read the data downloaded from the sensing host, and then use the software to convert the data into a CSV file 102 with the gas chromatography VOC values. The automatic data correction module 20 can read the CSV file 102 of the gas chromatography VOC values, perform drift correction on the data of the CSV file 102, and export the atmospheric VOC species concentration report 50 with correct concentration value.

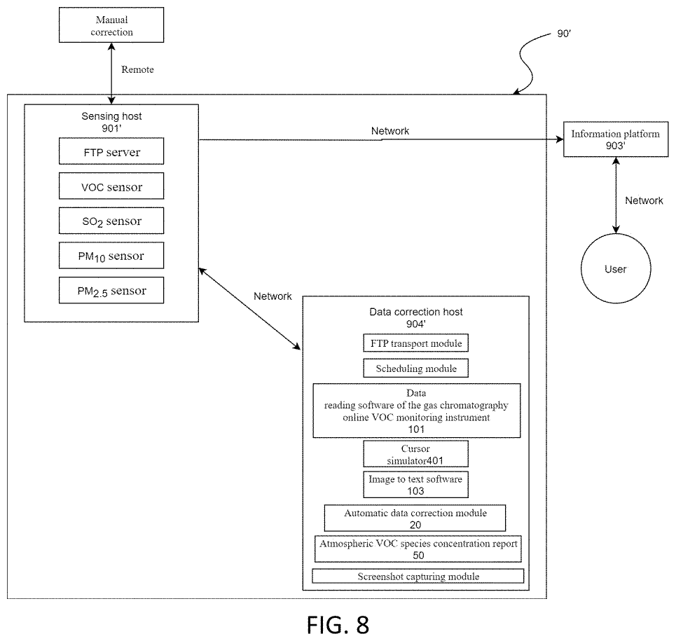

FIG. 8 shows a structural view of an unmanned automatic data correction system of the gas mass spectrometry online VOC monitor 90′ of a fifth practical use example. The sensing host 901′ is mainly used to sense the various species concentrations in atmosphere, and generate various species concentration reports. The different between the gas chromatography online VOC monitoring system and the gas mass spectrometry online VOC monitoring system 90′ is that the gas mass spectrometry online VOC monitoring system 90′ is not provided with an industrial personal computer, and the sensing host 901′ directly uploads the data to the information platform 903′ and the client can query real-time generated data through the information platform 903′. However, the data generated by the sensing host 901′ may have data drift and must be corrected to ensure normality thereof. The manual correction manner is performed by remotely logging into the sensing host 901′ to capture data, and manually correcting the data and then transmitting the corrected data back to the sensing host 901′ which then uploads the corrected data to the information platform 903′ through network. The unmanned automatic data correction system of the gas mass spectrometry online VOC monitor 90′ additionally includes a data calibration host 904′ which is used to replace the manual correction. The data calibration host 904′ uses the FTP transport module to download the raw data, generated by the sensing host 901′, through network. The computer cursor simulator 401 of the data calibration host 904′ can simulate a person's operating behavior to use the data reading software 101 of the gas mass spectrometry online VOC monitor, to read the data downloaded from the sensing host 901′ or the information platform 903′. The different between the gas chromatography online VOC monitor and the gas mass spectrometry online VOC monitor is that the gas mass spectrometry online VOC monitor uses the screen capture program to capture a screenshot of the spectrum data generated by the data reading software 101 of gas mass spectrometry online VOC monitor, and the image to text software 103 converts the spectrum data into the data readable for the automatic data correction module 20. After the automatic data correction module 20 corrects the read data according to the preset standard spectrum or the preset algorithm, the correct atmospheric VOC species concentration report 50 is generated.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. A calibrating device for automatically calibrating data of measuring instrument, comprising:
a data reading and converting module configured to obtain readable data having time information and generated by a measuring instrument; and
an automatic data correction module configured to compare a preset standard spectrum with the obtained readable data having time information, and execute corresponding data correction according to a comparison result.

2. The calibrating device according to claim 1, further comprising:
a data report module configured to generate an instrument data report.

3. The calibrating device according to claim 1, further comprising:
an automatic operation module configured to control the measuring instrument to generate a instrument data report.

4. The calibrating device according to claim 1, wherein the data reading and converting module, which obtains the readable data having time information and generated by the measuring instrument, is operative to:
obtain raw data generated by the measuring instrument; and
when the raw data is a comma-separated value (CSV) file, use the CSV file as the readable data having time information.

5. The calibrating device according to claim 1, wherein the data reading and converting module, which obtains the readable data having time information and generated by the measuring instrument, is operative to:
obtain the raw data generated by the measuring instrument; and
when the raw data is a CSV file but not directly readable, remove abnormal values in the CSV file, and use the CSV file with abnormal value removed as the readable data having time information.

6. The calibrating device according to claim 1, wherein the data reading and converting module, which obtains the readable data having time information and generated by the measuring instrument, is operative to:
obtain raw data generated by the measuring instrument; and
when the raw data is a screenshot of spectrum data generated by the measuring instrument, convert the screenshot into the readable data having time information.

7. The calibrating device according to claim 4, wherein the manner for the data reading and converting module to obtain the raw data generated by the measuring instrument, is one of following manners:
a manner of receiving, by the data reading and converting module, the raw data transmitted by the measuring instrument;
a manner of downloading, by the data reading and converting module, the raw data uploaded by the measuring instrument, from the industrial personal computer; and
a manner of downloading, by the data reading and converting module, the raw data uploaded by the measuring instrument, from the platform server.

8. The calibrating device according to claim 5, wherein the manner for the data reading and converting module to obtain the raw data generated by the measuring instrument, is one of following manners:
a manner of receiving, by the data reading and converting module, the raw data transmitted by the measuring instrument;
a manner of downloading, by the data reading and converting module, the raw data uploaded by the measuring instrument, from the industrial personal computer; and
a manner of downloading, by the data reading and converting module, the raw data uploaded by the measuring instrument, from the platform server.

9. The calibrating device according to claim 6, wherein the manner for the data reading and converting module to obtain the raw data generated by the measuring instrument, is one of following manners:
a manner of receiving, by the data reading and converting module, the raw data transmitted by the measuring instrument;
a manner of downloading, by the data reading and converting module, the raw data uploaded by the measuring instrument, from the industrial personal computer; and
a manner of downloading, by the data reading and converting module, the raw data uploaded by the measuring instrument, from the platform server.

10. The calibrating device according to claim 4, further comprising:
an automatic operation module controlled by the automatic data correction module, to automatically activate the measuring instrument to generate the raw data.

11. The calibrating device according to claim 5, further comprising:
an automatic operation module controlled by the automatic data correction module, to automatically activate the measuring instrument to generate the raw data.

12. The calibrating device according to claim 6, further comprising:
an automatic operation module controlled by the automatic data correction module, to automatically activate the measuring instrument to generate the raw data.

13. A calibrating method for automatically calibrating data of measuring instrument, comprising:
obtaining readable data having time information and generated by a measuring instrument;
comparing the preset standard spectrum with the readable data having time information, and executing corresponding data correction according to a comparison result.

14. The calibrating method according to claim 13 further comprising:
generating an instrument data report.

15. The calibrating method according to claim 13, further comprising:
controlling the measuring instrument to generate an instrument data report.

16. The calibrating method according to claim 13, wherein the step of obtaining the readable data having time information and generated by the measuring instrument, further comprises:
obtaining raw data generated by the measuring instrument; and
when the raw data is a CSV file, using the CSV file as the readable data having time information.

17. The calibrating method according to claim 13, wherein the step of obtaining the readable data having time information and generated by the measuring instrument, further comprising:
obtaining raw data generated by the measuring instrument;
when the raw data is a CSV file but not directly readable, removing abnormal values in the CSV file, using the CSV file with abnormal value removed as the readable data having time information.

18. The calibrating method according to claim 13, wherein the step of obtaining the readable data having time information and generated by the measuring instrument, further comprises:
obtaining the raw data generated by the measuring instrument;
when the raw data is a screenshot of spectrum data generated by the measuring instrument, converting the screenshot into the readable data having time information.

19. The calibrating method according to claim 16, wherein the step of obtaining the raw data generated by the measuring instrument is one of steps:
receiving the raw data transmitted from the measuring instrument;
downloading the raw data, which is uploaded by the measuring instrument, from an industrial personal computer; and
downloading the raw data, which is uploaded by the measuring instrument, from a platform server.

20. The calibrating method according to claim 17, wherein the step of obtaining the raw data generated by the measuring instrument is one of steps:
receiving the raw data transmitted from the measuring instrument;
downloading the raw data, which is uploaded by the measuring instrument, from an industrial personal computer; and
downloading the raw data, which is uploaded by the measuring instrument, from a platform server.

21. The calibrating method according to claim 18, wherein the step of obtaining the raw data generated by the measuring instrument is one of steps:
receiving the raw data transmitted from the measuring instrument;
downloading the raw data, which is uploaded by the measuring instrument, from an industrial personal computer; and
downloading the raw data, which is uploaded by the measuring instrument, from a platform server.

22. The method according to claim 16, further comprising:
controlling the measuring instrument to automatically start generating the raw data.

23. The method according to claim 17, further comprising:
controlling the measuring instrument to automatically start generating the raw data.

24. The method according to claim 18, further comprising:
controlling the measuring instrument to automatically start generating the raw data.
